(19) United States
(12) Patent Application Publication
(10) Pub. No.: US 2024/0343362 A1
(43) Pub. Date: Oct. 17, 2024

(54) DIVING MASK

(71) Applicant: Dongguan Kuyou Sports Goods Co., LTD, Dongguan (CN)

(72) Inventor: Zhiyuan Lin, Shantou (CN)

(21) Appl. No.: 18/314,382

(22) Filed: May 9, 2023

(30) Foreign Application Priority Data
Apr. 13, 2023 (CN) 202320815136.2

(51) Int. Cl. B63C 11/12; F41B 9/00

(52) U.S. Cl. CPC B63C 11/12; A61F 9/029; F41B 9/004

(57) ABSTRACT

The present disclosure discloses a diving mask, including a mask assembly, a water suction pump, a suction pipe, and a water outlet pipe. The mask assembly is provided with a water spraying hole and an accommodating cavity. The water suction pump is arranged on the mask assembly. The water suction pump is provided with a piston end, a suction end, and a water outlet end. The suction end is communicated with the water outlet end. The piston end is used for making the suction end to pump water. One end of the suction pipe is communicated with the suction end. One end of the water outlet pipe is communicated with the water outlet end, and the other end is communicated with the water spraying hole. The diver can shoot with sprayed water without wearing an additional water gun, which can improve the entertainment of the diving mask.

### TECHNICAL FIELD

The present disclosure relates to the technical field of diving supplies, in particular to, a diving mask.

### BACKGROUND

As is well known, a traditional diving mask has a relatively single function. During diving, a diver needs to wear an additional water gun, to play with companions by spraying water. However, wearing an additional water gun easily increases the diving burden for a diver, and the diver needs to hold the water gun with both hands to aim and shoot, making it inconvenient for the diver to shoot with sprayed water. The diving mask is low in entertainment.

### SUMMARY

The present disclosure aims to at least solve one of the technical problems in the existing art. To this end, the present disclosure provides a diving mask, which can improve the entertainment of the diving mask.

The diving mask according to an embodiment of the present disclosure includes a mask assembly, a water suction pump, a suction pipe, and a water outlet pipe. The mask assembly is provided with a water spraying hole and an accommodating cavity; the water suction pump is arranged on the mask assembly; the water suction pump is provided with a piston end, a suction end, and a water outlet end; the suction end is communicated with the water outlet end; the piston end is used for making the suction end to pump water; one end of the suction pipe is communicated with the suction end; and one end of the water outlet pipe is communicated with the water outlet end, and the other end is communicated with the water spraying hole.

The diving mask according to an embodiment of the present disclosure at least has the following beneficial effects:

To shoot with sprayed water, the diver can press the piston end to discharge air in the suction pipe, so that an air pressure in the suction pipe is less than an external atmospheric pressure, making the suction pipe pump water. The water pumped in by the suction pipe flows to the water outlet end via the suction end, then flows to the water spraying hole through the water outlet pipe, and is sprayed to the outside. The diver can shoot with sprayed water without wearing an additional water gun, which can reduce the diving burden of the diver and improve the entertainment of the diving mask.

According to some embodiments of the present disclosure, the mask assembly is provided with a first accommodating channel, and the water outlet pipe is arranged in the first accommodating channel in a penetrating manner.

According to some embodiments of the present disclosure, the mask assembly is provided with a fixing hole, and the suction pipe is arranged in the fixing hole in a penetrating manner.

According to some embodiments of the present disclosure, the mask assembly is provided with a second accommodating channel; the second accommodating channel is communicated with the fixing hole; and the suction pipe is arranged in the second accommodating channel in a penetrating manner.

According to some embodiments of the present disclosure, the water spraying hole is formed in an upper end of the mask assembly, and the fixing hole is formed in a lower end of the mask assembly.

According to some embodiments of the present disclosure, the mask assembly is provided with a mounting portion; the mounting portion is used to be connected to a water storage bottle; and one end of the suction pipe far away from the water suction pump is inserted into the water storage bottle.

According to some embodiments of the present disclosure, a diameter of the water spraying hole is less than a diameter of the water outlet pipe.

According to some embodiments of the present disclosure, the water suction pump includes a pump body assembly, a piston member, a first blocking piece, and a second blocking piece; the pump body assembly is provided with a first channel and a second channel; a middle position of the second channel is communicated with the first channel; the piston member is accommodated in the first channel, and can move along an axial direction of the first channel; the piston end is arranged at the piston member; the suction end and the water outlet end are respectively arranged at two ends of the second channel; the first blocking piece and the second blocking piece are movably arranged in the second channel in a penetrating manner; the first blocking piece is used for opening and blocking the suction end; and the second blocking piece is used for opening and blocking the water outlet end.

According to some embodiments of the present disclosure, the diving mask further includes a pressing member; the mask assembly is provided with an avoiding port; the pressing member is movably arranged in the avoiding port in a penetrating manner and is connected with the piston member; and the pressing member is used for pressing the piston member.

According to some embodiments of the present disclosure, the diving mask further includes a breather assembly; the breather assembly is provided with an air inlet channel and a first air outlet channel which are spaced apart from each other; the mask assembly is provided with a second air outlet channel arranged along a circumferential direction of the accommodating cavity; a wall surface of the accommodating cavity is provided with a first air inlet and an air outlet; the first air inlet is communicated with the air inlet channel and is located on an upper side of the accommodating cavity; the second air outlet channel is communicated with the first air outlet channel and the air outlet; and the air outlet is located on a lower side of the accommodating cavity.

Additional aspects and advantages of the present disclosure will be provided in the following descriptions, and will become apparent from the following descriptions or be learned through the practice of the present disclosure.

### BRIEF DESCRIPTION OF THE DRAWINGS

The above and/or additional aspects and advantages of the present disclosure will become apparent and easily understandable from the following descriptions of the embodiments with reference to the accompanying drawings.

FIG. 1 is a schematic structural diagram of a diving mask in an embodiment of the present disclosure;

FIG. 2 is a schematic structural diagram of a diving mask in an embodiment of the present disclosure in another direction;

FIG. 3 is a schematic diagram of an internal structure of a diving mask in an embodiment of the present disclosure;

FIG. 4 is a partially enlarged diagram of the part A in FIG. 3;

FIG. 5 is a schematic exploded diagram of a diving mask in an embodiment of the present disclosure;

FIG. 6 is a schematic structural diagram of a first accommodating channel and a second accommodating channel in an embodiment of the present disclosure;

FIG. 7 is a schematic structural diagram of a second air outlet channel and an accommodating cavity in an embodiment of the present disclosure;

FIG. 8 is a schematic structural diagram of a water suction pump in an embodiment of the present disclosure;

FIG. 9 is a schematic diagram of an internal structure of a water suction pump in an embodiment of the present disclosure; and

FIG. 10 is a schematic exploded diagram of a water suction pump in an embodiment of the present disclosure.

### REFERENCE NUMERALS IN THE DRAWINGS

mask assembly 100, water spraying hole 101, accommodating cavity 102, first accommodating channel 103, first fixing location 1031, fixing hole 104, second accommodating channel 105, second fixing location 1051, avoiding port 106, first air inlet 107, air outlet 108, second air outlet channel 109, mounting portion 110, frame 120, mask main body 130, shell 140, water suction pump 200, piston end 201, suction end 202, water outlet end 203, pump body assembly 210, first channel 211, second channel 212, piston member 220, first blocking piece 230, second blocking piece 240, reset member 250, pressing member 300, breather assembly 400, floating ball member 401, anti-suffocation hole 4011, air inlet channel 410, first channel 411, second channel 412, third channel 413, third air inlet 4131, second air inlet 4132, and first air outlet channel 420.

### DETAILED DESCRIPTION OF THE EMBODIMENTS

The embodiments of the present disclosure are described in detail below. Examples of the embodiments are shown in the accompanying drawings. The same or similar reference signs represent the same or similar elements or elements having the same or similar functions throughout. The embodiments described below with reference to the drawings are exemplary, and are only used to explain the present disclosure, and should not be construed as limiting the present disclosure.

In the description of the present disclosure, it should be understood that orientations or positional relationships indicated by the terms “upper”, “lower”, “front”, “rear”, “left”, “right”, and the like are orientations or positional relationships as shown in the drawings, and are only for the purpose of facilitating and simplifying the description of the present disclosure instead of indicating or implying that devices or elements indicated must have particular orientations, and be constructed and operated in the particular orientations, so that these terms are not construed as limiting the present disclosure.

In the description of the present disclosure, “several” means one or more; “multiple” means two or more; “greater than”, “less than”, “exceeding”, and the like mean not including this number; and “above”, “below”, and “within” are understood as including this number. If it is described that first and second are only for the purpose of distinguishing the technical features, and cannot be understood as indicating or implying the relative importance or implicitly specifying the number of the indicated technical features or implicitly specifying the order of the indicated technical features.

In the description of the present disclosure, unless otherwise specified, terms such as arrange, mount, and connect should be understood in a broad sense, and those skilled in the art can reasonably determine the specific meanings of the above terms in the present disclosure in combination with the specific content of the technical solution.

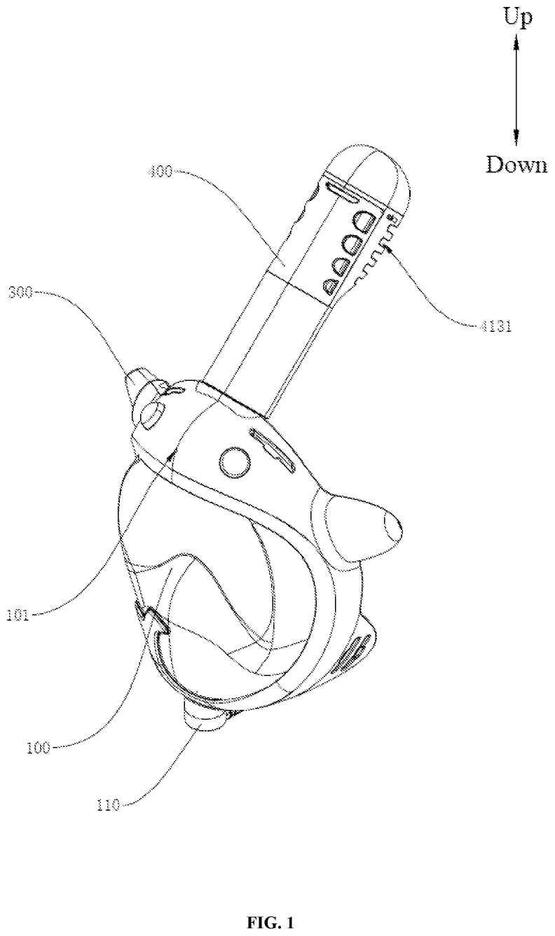

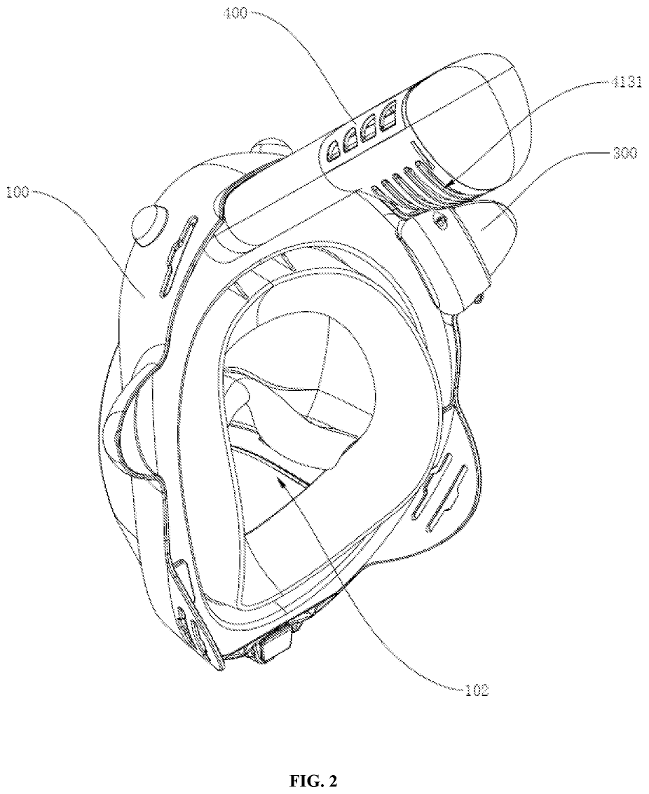

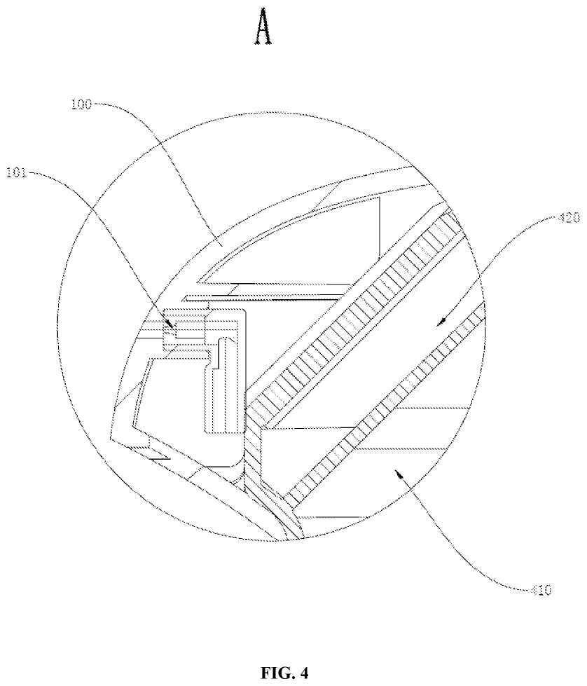

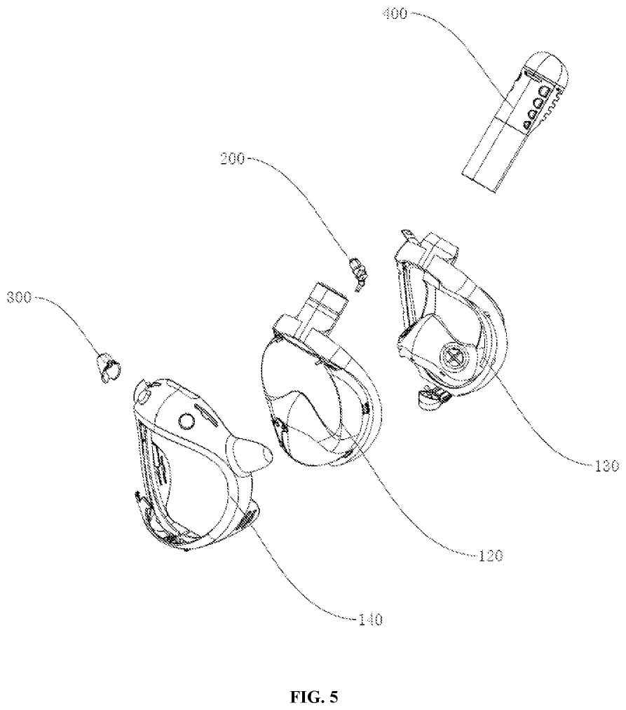

Referring to FIG. 1 to FIG. 5, a diving mask according to an embodiment of the present disclosure includes a mask assembly 100, a water suction pump 200, a suction pipe (not shown), and a water outlet pipe (not shown). The mask assembly 100 is provided with a water spraying hole 101 and an accommodating cavity 102. The water suction pump 200 is arranged on the mask assembly 100. The water suction pump 200 is provided with a piston end 201, a suction end 202, and a water outlet end 203. The suction end 202 is communicated with the water outlet end 203. The piston end 201 is used for making the suction end 202 to pump water. One end of the suction pipe is communicated with the suction end 202. One end of the water outlet pipe is communicated with the water outlet end 203, and the other end is communicated with the water spraying hole 101. In this way, a diver can press the piston end 201, making the suction end 202 pump water. The water pumped in by the suction end 202 can be discharged to the water spraying hole 101 from the water outlet end 203 and sprayed to the outside. The diver can shoot with sprayed water without wearing an additional water gun, which can improve the entertainment of the diving mask.

Specifically, when the diver shoots with sprayed water, the diver can press the piston end 201 to discharge air in the suction pipe, so that an air pressure in the suction pipe is less than an external atmospheric pressure, making the suction pipe pump water. The water pumped in by the suction pipe flows to the water outlet end 203 via the suction end 202, then flows to the water spraying hole 101 through the water outlet pipe, and is sprayed to the outside. The diver can shoot with sprayed water without wearing an additional water gun, which can reduce the diving burden of the diver and improve the entertainment of the diving mask.

It should be noted that the accommodating cavity 102 is used for accommodating the head of the diver, which will not be limited here.

In some embodiments of the present disclosure, the mask assembly 100 includes a frame 120, a mask main body 130, and a shell 140. The frame 120 is connected with the shell 140, and the mask main body 130 is sandwiched between the frame 120 and the shell 140. The mask main body 130 is enclosed with the frame 120 to form the accommodating cavity 102, which can facilitate fixing the mask main body 130.

It should be noted that the frame 120 and the shell 140 can be connected in a bolted manner or through a clamping structure, as long as the manner is convenient for assembling the frame 120 and the shell 140, which will not be limited here.

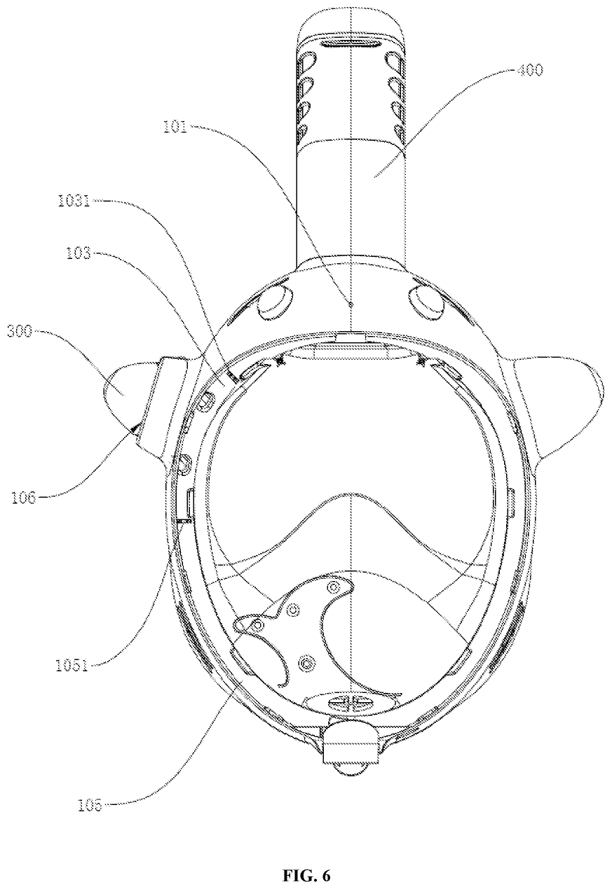

Referring to FIG. 6, in some embodiments of the present disclosure, the mask assembly 100 is provided with a first accommodating channel 103, and the water outlet pipe is arranged in the first accommodating channel 103 in a penetrating manner, so that the water outlet pipe can be hidden, to avoid the water outlet pipe from being exposed, so as to reduce the overall volume of the diving mask and facilitate package and transportation of the diving mask.

Specifically, the first accommodating channel 103 is arranged along a circumferential direction of the shell 140, and the water outlet pipe is arranged in the first accommodating channel 103 in the penetrating manner, so that the water outlet pipe can be hidden, to avoid the water outlet pipe from being exposed, so as to reduce the overall volume of the diving mask and facilitate package and transportation of the diving mask.

It should be noted that a wall surface of the first accommodating channel 103 is provided with a first fixing location 1031. The first fixing location 1031 is used for fixing the water outlet pipe. The first fixing location 1031 is a fastener structure, which will not be described in detail here.

It should be noted that the water outlet pipe is made of a flexible material, which can bend and deform and will not be limited here.

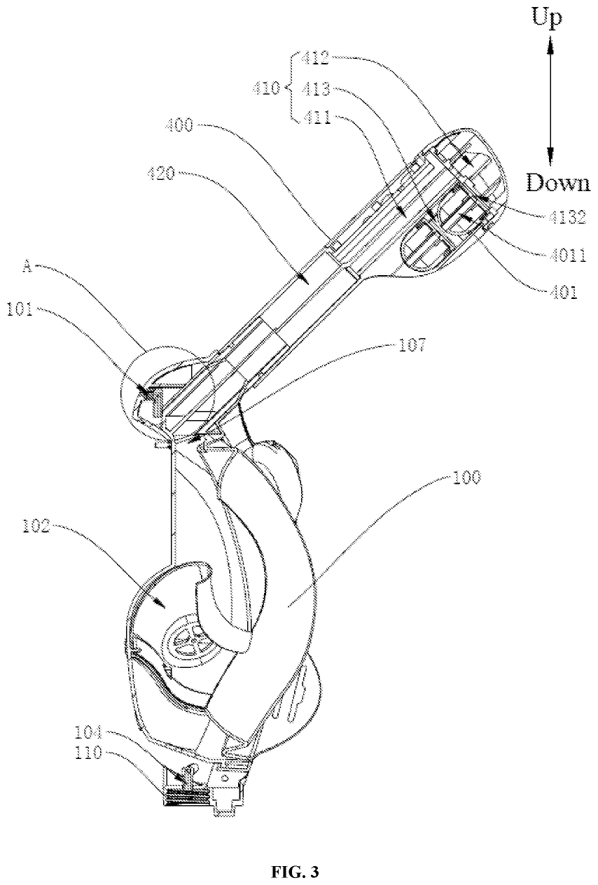

Referring to FIG. 3, in some embodiments of the present disclosure, the mask assembly 100 is provided with a fixing hole 104. The suction pipe is arranged in the fixing hole 104 in a penetrating manner, so that the suction pipe can be conveniently fixed, to facilitate the water suction pump 200 to pump water.

Specifically, the suction pipe is arranged in the fixing hole 104 in the penetrating manner, so that one end of the suction pipe far away from the water suction pump 200 cannot freely swing. The suction pipe can be conveniently fixed, to facilitate the water suction pump 200 to pump water.

Referring to FIG. 6, in some embodiments of the present disclosure, the mask assembly 100 is provided with a second accommodating channel 105; the second accommodating channel 105 is communicated with the fixing hole 104; and the suction pipe is partially accommodated in the second accommodating channel 105, so that the suction pipe can be hidden, to avoid the suction pipe from being exposed, so as to reduce the overall volume of the diving mask and facilitate package and transportation of the diving mask.

Specifically, the second accommodating channel 105 is arranged along the circumferential direction of the shell 140, and the suction pipe is arranged in the second accommodating channel 105 in the penetrating manner, so that the suction pipe can be hidden, to avoid the suction pipe from being exposed, so as to reduce the overall volume of the diving mask and facilitate package and transportation of the diving mask.

It should be noted that a wall surface of the second accommodating channel 105 is provided with a second fixing location 1051. The second fixing location 1051 is used for fixing the suction pipe. The second fixing location 1051 is a fastener structure, which will not be described in detail here.

It should be noted that the suction pipe is made of a flexible material, which can bend and deform and will not be limited here.

Referring to FIG. 6, in some embodiments of the present disclosure, the water spraying hole 101 is formed in an upper end of the mask assembly 100, and the fixing hole 104 is formed in a lower end of the mask assembly 100, to facilitate the diver to shoot with sprayed water.

Specifically, the water spraying hole 101 is formed in the upper end of the mask assembly 100, and the fixing hole 104 is formed in the lower end of the mask assembly 100. To shoot with sprayed water, the diver floats up slightly, making the water spraying hole 101 exposed from water, and the diver then presses the piston end 201 to complete shooting with sprayed water. The diver can conveniently shoot with sprayed water without completely raising the head above water.

Referring to FIG. 6, in some embodiments of the present disclosure, the mask assembly 100 is provided with a mounting portion 110. The mounting portion 110 is used to be connected with a water storage bottle which is used for storing water. The end of the suction pipe far away from the water suction pump 200 can be inserted into the water storage bottle. By the arrangement of the mounting portion 110, the water storage bottle is convenient to mount.

Specifically, the mounting portion 110 is an internal thread structure which can be in threaded connection with an external thread structure at a bottleneck of the water storage bottle, to facilitate mounting and removal of the water storage bottle.

It should be noted that the mounting portion 110 may also be an external thread structure, and the bottleneck of the water storage bottle is provided with an internal thread structure that is in threaded connection with the external thread structure. This will not be limited here.

Of course, in some specific implementations, the mounting portion 110 may also be replaced with a clamping portion which may be a fastener or a fastening slot, as long as it can be in clamping fit with the water storage bottle. This will not be limited here.

In some embodiments of the present disclosure, a diameter of the water spraying hole 101 is less than a diameter of the water outlet pipe, so that the water spraying hole 101 can pressurize water in the water outlet pipe, and the water spraying hole 101 sprays water at a longer spraying distance, which can improve the user experience and improve the market competitiveness of the diving mask.

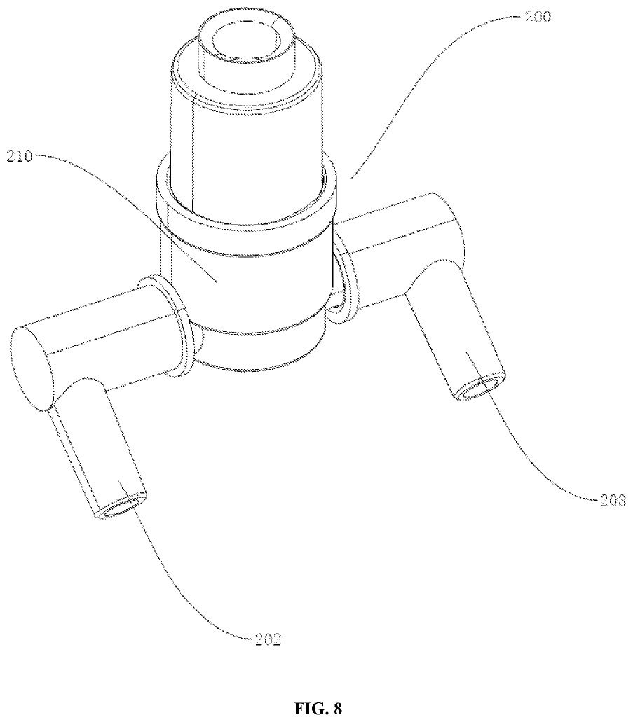

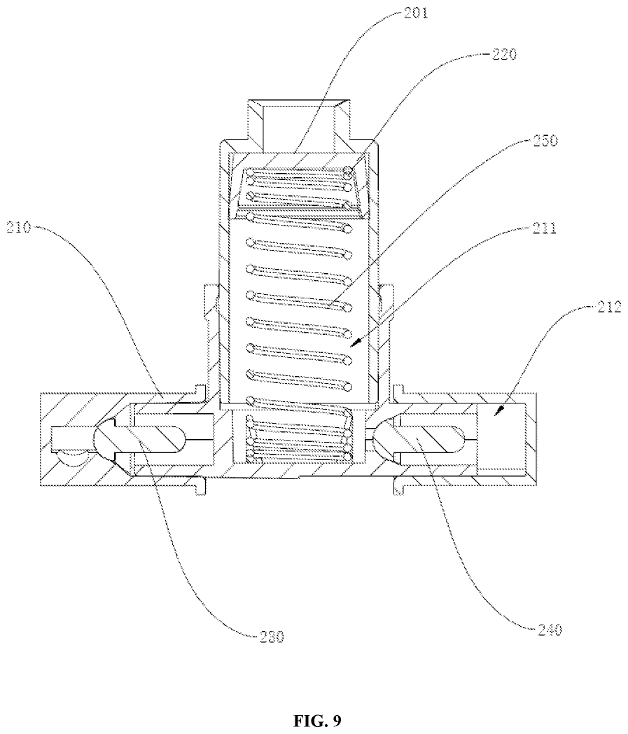

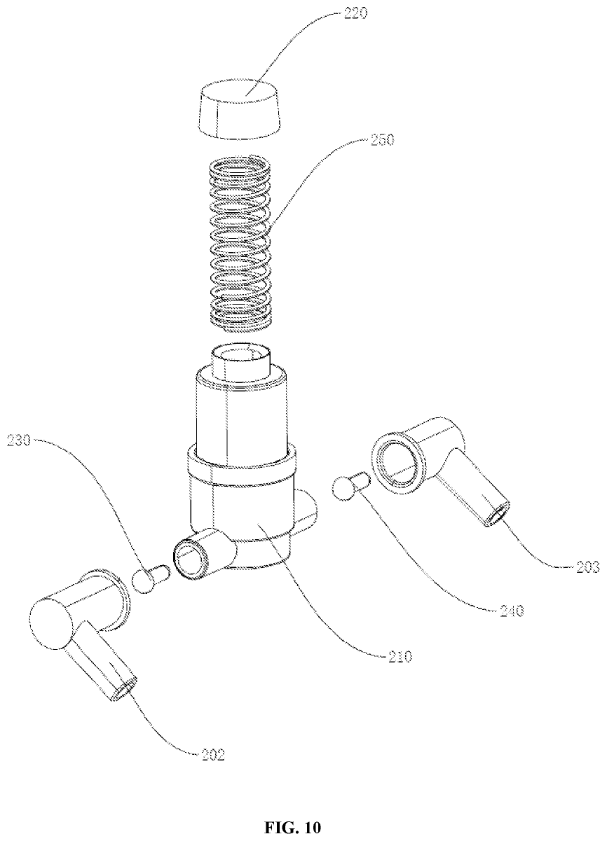

Referring to FIG. 8, FIG. 9, and FIG. 10, in some embodiments of the present disclosure, the water suction pump 200 includes a pump body assembly 210, a piston member 220, a first blocking piece 230, and a second blocking piece 240. The pump body assembly 210 is provided with a first channel 211 and a second channel 212. A middle position of the second channel 212 is communicated with the first channel 211. The piston member 220 is accommodated in the first channel 211, and can move along an axial direction of the first channel 211. The suction end 202 and the water outlet end 203 are respectively arranged at two ends of the second channel 212. The first blocking piece 230 and the second blocking piece 240 are movably arranged in the second channel 212 in a penetrating manner. The first blocking piece 230 is used for opening and blocking the suction end 202. The second blocking piece 240 is used for opening and blocking the water outlet end 203. It can be convenient for the water suction pump 200 to pump water, and the diver can conveniently shoot with sprayed water.

Specifically, to shoot with sprayed water, the diver can press the pressing member 300 to cause the piston member 220 to move towards the second channel 212, to discharge air in the first channel 211. The discharged air pushes the first blocking piece 230 to block the suction end 202, and also pushes the second blocking piece 240 to open the water outlet end 203, so that the air in the first channel 211 is discharged via the water outlet hole 101. The piston member 220 is then caused to move away from the second channel 212. As an air pressure in the first channel 211 decreases, water in the suction pipe can enter the first channel. The water is then pumped away by the water suction pump 200 and is sprayed out from the water outlet hole 101, thus completing the shooting action with sprayed water. The operation is simple, and it can be convenient for the diver to shoot with sprayed water, to improve the entertainment of the diving mask.

It should be noted that the piston member 220 abuts against a side wall of the first channel 211, so that the air in the first channel 211 can only be discharged from the second channel 212, which will not be described in detail.

It should be noted that a reset member 250 is arranged between the piston member 220 and the pump body assembly 210. One end of the reset member 250 is connected with the piston member 220, and the other end is connected with the pump body assembly 210, so that the reset member 250 can actuate the piston member 220 to move away from the second channel 212, and the piston member 220 can be automatically reset, to facilitate the diver to repeat the water spraying design and improve the use experience of the diver.

It should be noted that the reset member 250 is a compression spring, a rubber member, or other structures capable of undergoing axial elastic deformation, which will not be limited here.

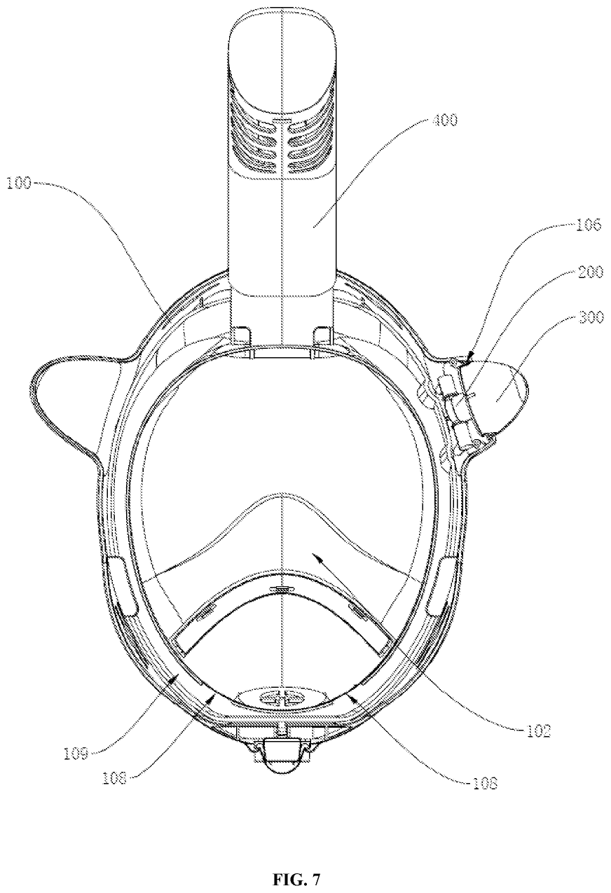

Referring to FIG. 7 and FIG. 8, in some embodiments of the present disclosure, the diving mask further includes a pressing member 300. The mask assembly 100 is provided with an avoiding port 106. The pressing member 300 is movably arranged in the avoiding port 106 in a penetrating manner and is connected with the piston member 220. The pressing member 300 is used for pressing the piston member 220, so that it can be convenient for the diver to shoot with sprayed water.

Specifically, the pressing member 300 is arranged in the avoiding port 106 in the penetrating manner. One end of the pressing member 300 is connected with the piston member 220, and the other end of the pressing member 300 extends out of an outer side of the mask assembly 100. A moving direction of the pressing member 300 is parallel to a moving direction of the piston member 220. It can be convenient for the diver to press the piston member 220, and the diver can shoot with sprayed water.

Referring to FIG. 3, in some embodiments of the present disclosure, the diving mask further includes a breather assembly 400. The breather assembly 400 is provided with an air inlet channel 410 and a first air outlet channel 420 which are spaced apart from each other. The frame 120 is provided with a first air inlet 107, an air outlet 108, and a second air outlet channel 109 arranged along a circumferential direction of the accommodating cavity 102. The air inlet channel 410 is communicated with the accommodating cavity 102 through the first air inlet 107. The second air outlet channel 109 is communicated with the first air outlet channel 420 and the air outlet 108 respectively. In this way, fresh air in the air inlet channel 410 can be avoided from being mixed with exhaled exhaust gas in the first air outlet channel 420, to ensure that a concentration of oxygen in the accommodating cavity 102 is within a normal range, thereby avoiding the chest tightness of the diver.

Specifically, the air inlet channel includes a first channel 411, a second channel 412, and a third channel 413. The first channel 411 and the third channel 413 are arranged side by side. One end of the second channel 412 is communicated with the first channel 411, and the other end is communicated with the third channel 413. The first channel 411, the second channel 412, and the third channel 413 are connected in sequence to form a U-shaped structure. A third air inlet 4131 is formed in a side wall of the third channel 413. Fresh air enters the third channel 413 via the third air inlet 4131, and then enters the accommodating cavity 102 through the second channel 412, the first channel 411, and the first air inlet 107 in sequence. One end of the first air outlet channel 420 away from the air outlet 108 is communicated to the outside. Exhaled exhaust gas in the accommodating cavity 102 is discharged into the first air outlet channel 420 via the air outlet 108 and is discharged to the outside. The air inlet channel 410 and the first air outlet channel 420 are spaced apart, so that the fresh air in the air inlet channel 410 will not be mixed with the exhaled exhaust gas in the first air outlet channel 420, which can ensure that the concentration of the oxygen in the accommodating cavity 102 is within the normal range, thereby avoiding the chest tightness of the diver.

In addition, a second air inlet 4132 is formed in a connection between the third channel 413 and the second channel 412. The breather assembly 400 includes a floating ball member 401. The floating ball member 401 is accommodated in the third channel 413, and can move along an axial direction of the third channel 413. The floating ball member 401 is used for blocking or opening the second air inlet 4132. The floating ball member 401 is provided with an anti-suffocation hole 4011 along an axial direction of the floating ball member. The anti-suffocation hole 4011 penetrates through the floating ball member 401 to avoid the phenomenon that a diver suffocates, thereby improving the use reliability of the diving mask.

Specifically, when the diver dives, the floating ball member 401 may possibly abut against the circumferential edge of the second air inlet 4132, and the floating ball member 401 cannot be automatically separated from the circumferential edge of the second air inlet 4132 under its gravity. As a result, fresh air cannot be supplemented into the accommodating cavity 102 via the air inlet channel 410, and the diver suffocates. By the arrangement of the anti-suffocation hole 4011, even if the floating ball member 401 abuts against the circumferential edge of the second air inlet 4132, the external fresh air can still enter the air inlet channel 410 through the anti-suffocation hole 4011 to supply oxygen to the diver, which can avoid the phenomenon that the diver suffocates, thereby improving the use reliability of the diving mask.

It should be noted that a diameter of the anti-suffocation hole 4011 is 1 mm to 3 mm. When the diameter of the anti-suffocation hole 4011 is less than 1 mm, the too small diameter of the anti-suffocation hole 4011 cannot quickly supplement fresh air for the diver, and there is a risk that the diver feels tight in the chest. When the diameter of the anti-suffocation hole 4011 is greater than 3 mm, the too large diameter of the anti-suffocation hole 4011 makes the seawater flow into the air inlet channel 410 through the anti-suffocation hole 4011, and the diver easily inhales water. The diameter of the anti-suffocation hole 4011 is set to be 1 mm to 3 mm, so that the anti-suffocation hole 4011 can supplement fresh air for the diver quickly, and can also prevent the seawater from flowing into the air inlet channel 410. This can improve the use reliability of the diving mask.

It should be noted that the diameter of the anti-suffocation hole 4011 can be specifically 1 mm, 1.5 mm, 2 mm, 2.5 mm, and 3 mm, which will not be limited here.

The technical features of the embodiments described above can be arbitrarily combined. In order to make the description concise, all possible combinations of various technical features in the above embodiments are not completely described. However, the combinations of these technical features should be considered as the scope described in this specification as long as there is no contradiction in them.

The above describes this embodiment in detail in combination with the accompanying drawings, but the present disclosure is not limited to the above-mentioned embodiments. Various changes can be further made within the knowledge scope of those ordinarily skilled in the art and without departing from this concept.

What is claimed is:

1. A diving mask, comprising:
a mask assembly (100), which is provided with a water spraying hole (101) and an accommodating cavity (102);
a water suction pump (200), which is arranged on the mask assembly (100), wherein the water suction pump (200) is provided with a piston end (201), a suction end (202), and a water outlet end (203); the suction end (202) is communicated with the water outlet end (203); and the piston end (201) is used for making the suction end (202) to pump water;
a suction pipe, which has one end communicated with the suction end (202); and
a water outlet pipe, which has one end communicated with the water outlet end (203) and the other end communicated with the water spraying hole (101).

2. The diving mask according to claim 1, wherein the mask assembly (100) is provided with a first accommodating channel (103), and the water outlet pipe is arranged in the first accommodating channel (103) in a penetrating manner.

3. The diving mask according to claim 1, wherein the mask assembly (100) is provided with a fixing hole (104), and the suction pipe is arranged in the fixing hole (104) in a penetrating manner.

4. The diving mask according to claim 3, wherein the mask assembly (100) is provided with a second accommodating channel (105); the second accommodating channel (105) is communicated with the fixing hole (104); and the suction pipe is arranged in the second accommodating channel (105) in a penetrating manner.

5. The diving mask according to claim 3, wherein the water spraying hole (101) is formed in an upper end of the mask assembly (100), and the fixing hole (104) is formed in a lower end of the mask assembly (100).

6. The diving mask according to claim 3, wherein the mask assembly (100) is provided with a mounting portion (110); the mounting portion (110) is used to be connected to a water storage bottle; and one end of the suction pipe far away from the water suction pump (200) is inserted into the water storage bottle.

7. The diving mask according to claim 1, wherein a diameter of the water spraying hole (101) is less than a diameter of the water outlet pipe.

8. The diving mask according to claim 1, wherein the water suction pump (200) comprises a pump body assembly (210), a piston member (220), a first blocking piece (230), and a second blocking piece (240); the pump body assembly (210) is provided with a first channel (211) and a second channel (212); a middle position of the second channel (212) is communicated with the first channel (211); the piston member (220) is accommodated in the first channel (211), and moves along an axial direction of the first channel (211); the piston end (201) is arranged at the piston member (220); the suction end (202) and the water outlet end (203) are respectively arranged at two ends of the second channel (212); the first blocking piece (230) and the second blocking piece (240) are movably arranged in the second channel (212) in a penetrating manner; the first blocking piece (230) is used for opening and blocking the suction end (202); and the second blocking piece (240) is used for opening and blocking the water outlet end (203).

9. The diving mask according to claim 8, further comprising a pressing member (300), wherein the mask assembly (100) is provided with an avoiding port (106); the pressing member (300) is movably arranged in the avoiding port (106) in a penetrating manner and is connected with the piston member (220); and the pressing member (300) is used for pressing the piston member (220).

10. The diving mask according to claim 1, further comprising a breather assembly (400), wherein the breather assembly (400) is provided with an air inlet channel (410) and a first air outlet channel (420) which are spaced apart from each other; the mask assembly (100) is provided with a second air outlet channel (109) arranged along a circumferential direction of the accommodating cavity (102); a wall surface of the accommodating cavity (102) is provided with a first air inlet (107) and an air outlet (108); the first air inlet (107) is communicated with the air inlet channel (410) and is located on an upper side of the accommodating cavity (102); the second air outlet channel (109) is communicated with the first air outlet channel (420) and the air outlet (108); and the air outlet (108) is located on a lower side of the accommodating cavity (102).
